(19) United States
(12) Patent Application Publication
(10) Pub. No.: US 2025/0205042 A1
(43) Pub. Date: Jun. 26, 2025

(54) INTRAOCULAR LENS WITH ADJUSTABLE DIOPTER AFTER SURGERY

(71) Applicant: The Catholic University of Korea Industry-Academic Cooperation Foundation, Seoul (KR)

(72) Inventor: Dong Hyun Jee, Suwon-si (KR)

(21) Appl. No.: 18/989,631

(22) Filed: Dec. 20, 2024

(30) Foreign Application Priority Data
Dec. 21, 2023 (KR) 10-2023-0188976

(51) Int. Cl. A61F 2/16

(52) U.S. Cl. CPC A61F 2/1613

(57) ABSTRACT

An intraocular lens (IOL) with an adjustable diopter after surgery is disclosed. According to an aspect of the present disclosure, the intraocular lens with an adjustable diopter after surgery may include: a haptic with a cylindrical receptacle, wherein one or more fixtures are configured to extend outwardly on an outer surface of the receptacle; an optic mounted within the receptacle of the haptic, and configured to refract a light, wherein a thread is formed on an inner circumferential surface of the receptacle, and the optic is configured such that an outer circumferential surface thereof engages with the thread and rotates to be able to move forward or backward in a direction in which the light is incident in the receptacle; and one or more magnetic members provided externally in at least one location at a periphery of the optic to rotate the optic by magnetic force.

### CROSS-REFERENCE TO RELATED APPLICATION

This application claims priority to and the benefit of Korean Patent Application No. 10-2023-0188976, filed on Dec. 21, 2023, the disclosures of which are incorporated herein by reference in its entirety.

### TECHNICAL FIELD

The present disclosure relates to an intraocular lens (IOL) with an adjustable diopter after surgery, and more specifically, to an intraocular lens with an adjustable diopter in a non-invasive manner after the intraocular lens is surgically inserted into an eyeball.

### RELATED ART

Generally, there is a crystalline lens in a human eyeball that acts as a lens. This crystalline lens may become cloudy due to aging, external stimulation, and diseases, and the disease in which the crystalline lens becomes cloudy is called a cataract.

The fundamental treatment of the cataract is surgery, which mainly involves removing the existing cloudy cataract and inserting an intraocular lens manufactured artificially in its place.

FIG. 1 is a plan view illustrating an example of a conventional intraocular lens.

Referring to FIG. 1, the intraocular lens 10 includes a lens (optic) 20 configured to refract incident light to form an image of an external object on a retina and form a lens body, and a pair of fixtures (haptic) 30 connected in the form of arms at mutually symmetrical positions on a predetermined circumferential portion of the lens part 20.

That is, the fixtures 30 are fixed within the eyeball, and the lens 20 refracts the light incident on the eyeball to replace the role of the crystalline lens.

However, conventional intraocular lenses have the following problems.

In order to see objects clearly after cataract surgery, it is important to obtain a state of emmetropia in which the cornea and crystalline lens appropriately refract the light entering the eye and focus it exactly on the retinal surface, and in order to achieve this, it is important to determine exact diopter values (refractive powers) of the intraocular lens suitable for each individual patient's eye.

There are various calculations for determining the diopter values of such an intraocular lens, but due to various reasons, when an intraocular lens of a calculated refractive power index is inserted into an eyeball, the actual refractive power index may appear differently from when calculated, which leads to discomfort of the patient.

Unlike natural crystalline lenses, artificial intraocular lenses do not change their refractive power index. In other words, natural crystalline lenses adjust focus by thickening or thinning a capsular bag through contraction and relaxation of ciliary muscles, but artificial intraocular lenses do not have this ability to adjust focus.

Therefore, if the intraocular lens inserted through surgery does not fit well with the patient's vision or lifestyle, reoperation of the intraocular lens is technically more difficult, and the patient must wear glasses or live with discomfort despite undergoing intraocular lens surgery.

In addition, in the case of patients with astigmatism, the lens of the intraocular lens inserted into the eyeball may also be a toric lens, a lens for correcting astigmatism, but if the astigmatic axis of the lens inserted into the eye does not coincide with the astigmatic axis of the patient, the side effect of worsening astigmatism may occur. In addition, since conventional intraocular lenses once inserted into the eyeball cannot be corrected, there is no way to reverse it, and the patient must correct the astigmatic axis separately using glasses, which is inconvenient.

### SUMMARY

The present disclosure is intended to solve the above problems, and an object of the present disclosure is to provide an intraocular lens with an adjustable diopter after surgery, which can be adjusted in a non-invasive method even after insertion into the eyeball.

The objects of the present disclosure are not limited to the above-described objects, and other objects that are not mentioned will be able to be clearly understood by those skilled in the art to which the present disclosure pertains from the following description.

According to an aspect of the present disclosure, there is disclosed an intraocular lens (IOL) with an adjustable diopter after surgery, including: a haptic with a cylindrical receptacle, wherein one or more fixtures are configured to extend outwardly on an outer surface of the receptacle; an optic mounted within the receptacle of the haptic, and configured to refract a light, wherein a thread is formed on an inner circumferential surface of the receptacle, and the optic is configured such that an outer circumferential surface thereof engages with the thread and rotates to be able to move forward or backward in a direction in which the light is incident in the receptacle; and one or more magnetic members provided externally in at least one location at a periphery of the optic to rotate the optic by magnetic force.

The magnetic member may be a magnet or a magnet coated thereon.

The magnetic member may be a magnetic substance or a magnetic substance coated thereon.

The magnetic member may be configured to have a projected protrusion shape.

The IOL may further include a scale configured to indicate an angle by which the optic is rotated.

The scale may include a reference scale formed on the optic, and rotated together according to rotation of the optic to become a reference point; and rotation scales formed at a predetermined interval on a rim of the receptacle of the haptic to face the reference scale.

The rotation scale may be formed at intervals of 60 degrees.

The thread may be configured such that the optic is moved forward or backward by 1 mm when rotated 180 degrees in a normal or reverse direction.

The receptacle may include a stopper at a front end or a rear end thereof, and the stopper may extend inwardly of the receptacle to prevent the optic from being removed while rotating forward and backward.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a conventional general intraocular lens.

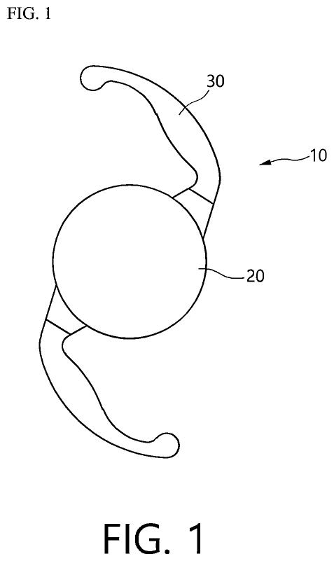

FIG. 2 is a view illustrating an intraocular lens with an adjustable diopter after surgery according to an embodiment of the present disclosure.

FIG. 3 is a cross-sectional view of an intraocular lens with an adjustable diopter after surgery according to an embodiment of the present disclosure.

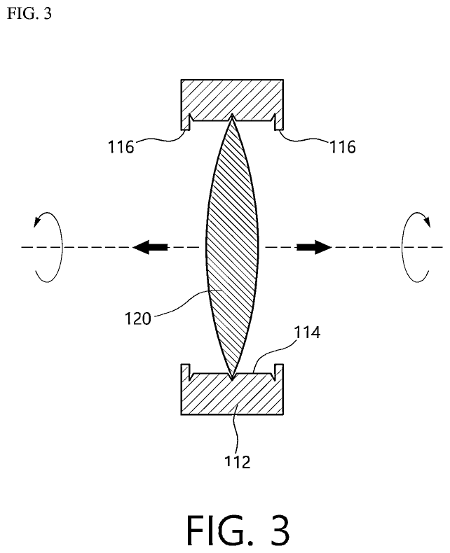

FIG. 4 is a view illustrating a state in which an intraocular lens with an adjustable diopter after surgery is inserted into an eyeball and then a diopter is adjusted according to an embodiment of the present disclosure.

FIG. 5 is a view illustrating a change in a position and a change in a diopter according to a rotation angle of an intraocular lens with an adjustable diopter after surgery according to an embodiment of the present disclosure.

### DETAILED DESCRIPTION OF THE EMBODIMENTS

Hereinafter, embodiments of the present disclosure will be described in detail so that those skilled in the art to which the present disclosure pertains can easily carry out the embodiments. The present disclosure may be implemented in many different forms and is not limited to the embodiments described herein. In order to clearly describe the present disclosure, portions not related to the description are omitted from the accompanying drawings, and the same or similar components are denoted by the same reference numerals throughout the specification.

The words and terms used in the specification and the claims are not limitedly construed as their ordinary or dictionary meanings, and should be construed as meaning and concept consistent with the technical spirit of the present disclosure in accordance with the principle that the inventors can define terms and concepts in order to best describe their invention. In the specification, it should be understood that the terms such as “comprise” or “have” are intended to specify the presence of features, numbers, steps, operations, components, parts, or combinations thereof described in the specification and do not preclude the possibility of the presence or addition of one or more other features, numbers, steps, operations, components, parts, or combinations thereof.

Hereinafter, an intraocular lens 100 with an adjustable diopter after surgery according to an embodiment of the present disclosure will be described with reference to the drawings.

The intraocular lens 100 with an adjustable diopter after surgery according to the present embodiment may include a haptic 110 and an optic 120, as shown in FIG.

2.

The optic 120 serves as a lens as a component for refracting light, and may be formed of a material through which light is transmitted. The optic 120 may use polymethyl methacrylate (PMMA) in the case of the hard intraocular lens 100, and may use silicone or acrylic materials in the case of the soft intraocular lens 100.

In addition, the optic 120 may generally take the form of a convex lens, but is not necessarily limited thereto, and may take the form of a circular cross-sectional shape.

The haptic 110 may be configured to mount the optic 120 and fix it within an eyeball.

The haptic 110 may include a receptacle 112 formed inside in the form of a cylinder to accommodate the optic 120, and a fixture 118 configured to extend outwardly on an outer surface of the receptacle 112 to be supported and fixed at any position in the eyeball. The fixture 118 may be provided in plurality.

In this case, the receptacle 112 of the haptic 110 may extend several mm in length toward the direction in which light is incident.

The haptic 110 may be made of a silicone material or an acrylic material having transparency, but is not limited thereto, and may be made of an opaque material if necessary.

In this case, a thread 114 is formed on an inner circumferential surface of the receptacle 112 of the haptic 110, and the optic 120 is configured such that an outer circumferential surface thereof engages with the thread 114 and rotates to be able to move forward or backward in a direction in which the light is incident in the receptacle 112.

To this end, an outer circumferential rim of the optic 120 may be configured to engage with the thread 114 formed in the receptacle 112.

In addition, a stopper 116 may be formed at a front end or a rear end of the receptacle 112 of the haptic 110, and the stopper 116 is configured to extend inwardly of the receptacle 112 to prevent the optic 120 from being removed from the receptacle 112 while rotating forward and backward.

In addition, the optic 120 may be provided with a magnetic member 122 externally so that the optic 120 may be rotated. Here, the external may mean the outside of the intraocular lens 100 inserted into the eyeball (e), that is, the outside of the eyeball (e), and may substantially mean the outside of the body.

The magnetic member 122 may be provided externally in at least one location at a periphery of the optic 120 so that the optic 120 may be rotated as a magnetic force.

At this time, the magnetic member 122 may be located at one or more locations on the circumference of the optic 120 that is not covered by the thread 114 of the receptacle 112 or the receptacle 112.

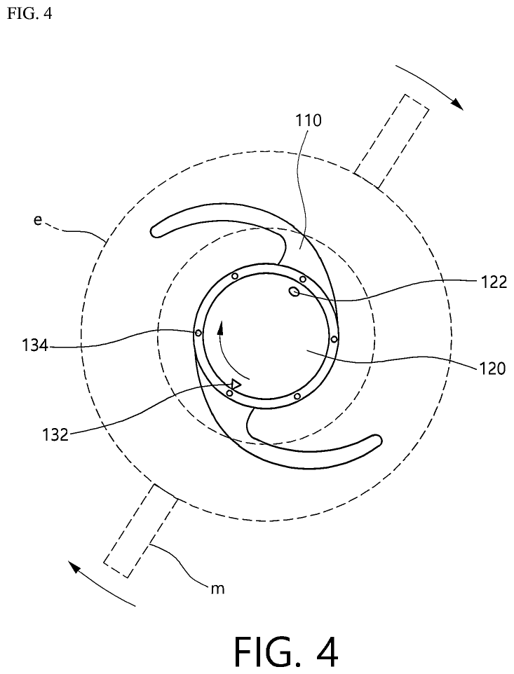

That is, as shown in FIG. 4, when the magnetic member 122 moves in a state in which a magnet (m) or a ferromagnetic material having a magnetic force or easily magnetized by the magnetic force is close to the outside of the eyeball (e), the magnetic member 122 may be forced as an attractive force or a repulsion force of the magnetic force to rotate the optic 120.

The magnetic member 122 may be a magnet or a magnet coated thereon.

Alternatively, the magnetic member 122 may be a magnetic substance or a magnetic substance coated thereon that is magnetized by the magnetic force of the outside and has magnetism, even if it is not the magnet (m).

The magnetic member 122 may be embedded in the optic 120 or may be provided to protrude to the outside of the optic 120. Of course, the magnetic member 122 may be coplanar with the surface of the optic 120.

In addition, the magnetic member 122 may be made of a material having excellent biocompatibility that does not cause rejection reactions or side effects when inserted into the human body, and may be, for example, neodymium magnet, nitinol, stainless steel, or the like.

As described above, as the optic 120 rotates, the position of the optic 120 moves forward or backward by the thread 114 formed on the inner surface of the receptacle 112 of the haptic 110, and as the position of the optic 120 moves, the actual diopter of the intraocular lens 100 may change.

For example, when the optic 120 rotates in the normal direction and moves 1 mm forward (to the side in which light is incident) by the threads 114, the diopter may increase by 3 diopters, and when the optic 120 rotates in the reverse direction and moves 1 mm backward by the threads 114, the diopter may decrease by −3 diopters.

Therefore, as shown in FIG. 4, when the optic 120 is rotated using the magnetic force such as the magnet (m) from the outside, the diopter of the intraocular lens 100 may be adjusted even when the intraocular lens 100 is inserted into the eyeball (e).

Meanwhile, the intraocular lens 100 with an adjustable diopter after surgery according to the present embodiment may further include a scale 130 configured to indicate an angle by which the optic 120 is rotated.

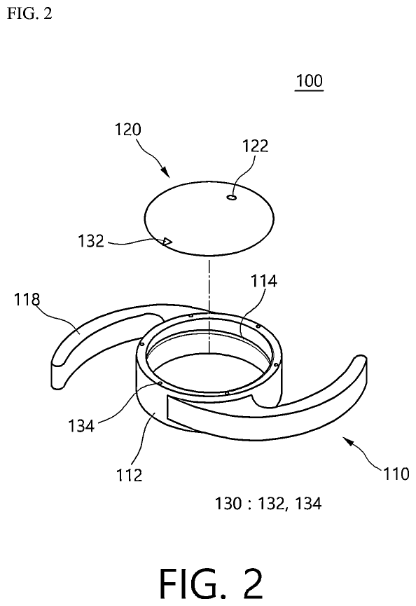

As shown in FIGS. 2 and 4, the scale 130 may include a reference scale 132 and a rotation scale 134.

The reference scale 132 is formed on the optic 120 and may be rotated together according to the rotation of the optic 120 to become a reference point for determining an angle at which the optic 120 is rotated.

The reference scale 132 may be engraved or embossed on the magnetic member 122, or may be formed at a separate location from the magnetic member 122 and may be displayed in a different color from the optic 120 so that an operator may check it.

The rotation scale 134 may be formed at regular intervals on the rim of the receptacle 112 of the haptic 110 to face the reference scale 132.

In the present embodiment, the rotation scale 134 is described as an example in which a total of six are arranged at intervals of 60 degrees. However, the rotation scale 134 may be arranged more densely than the intervals of 60 degrees or conversely, may be arranged at wider intervals.

In addition, in the present embodiment, the thread 114 may be configured such that the optic 120 is moved forward or backward 1 mm when rotated 180 degrees in a normal or reverse direction.

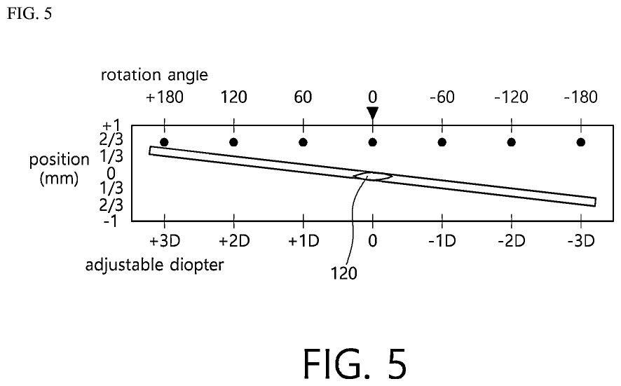

Therefore, as shown in FIG. 5, when the optic 120 is located in the center, the effect of refractive power change due to the position of the optic 120 is zero diopter, when the optic 120 is rotated 60 degrees in the normal direction, the optic 120 is moved forward by ⅓ mm, and the effect of refractive power change at this time is +1 diopter, when rotated 120 degrees, it is moved forward by ⅔ mm, and the effect of refractive power change at this time is +2 diopters, when rotated 180 degrees, it is moved forward by 1 mm, and the effect of refractive power change at this time can be +3 diopters.

In addition, on the contrary, when the optic 120 is rotated 60 degrees in the reverse direction, the optic 120 is moved backward by ⅓ mm and the effect of refractive power change at this time is −1 diopter, when rotated 120 degrees, it is moved backward by ⅔ mm and the effect of refractive power change at this time is −2 diopters, when rotated 180 degrees, it is moved backward by 1 mm and the effect of refractive power change at this time may be −3 diopters.

Therefore, after the intraocular lens implantation surgery, the patient may correct the diopter appropriate for their vision in a non-invasive manner while the intraocular lens 100 is inserted into the eyeball (e).

In addition, when there is no scale or index for the rotation angle of the intraocular lens, the operation of performing the patient's vision test after the insertion of the intraocular lens is completed, and the operation of performing the vision test again after adjusting the refractive power of the intraocular lens by the operator's sense are repeated until the patient's vision is properly adjusted, so there is a cost burden due to repeated surgeries, and both the operator and the patient are inconvenienced. But according to the intraocular lens with an adjustable diopter after surgery of the present disclosure, since the scale 130 having the index for the rotation angle of the optic 120 is provided, it is possible to determine how much the refractive power of the intraocular lens should be adjusted only once after the intraocular lens implantation surgery, and accordingly, the post-surgery vision correction may be completed by rotating the optic 120 by a predetermined angle.

In other words, since the vision correction can be completed with only one correction, convenience and satisfaction of the patient may be improved.

As described above, according to the intraocular lens with an adjustable diopter after surgery according to the present disclosure, since the diopter can be corrected in a non-invasive manner without surgery even after being inserted into the eyeball, the optimal diopter can be provided to the patient, thereby significantly improving the patient's satisfaction and quality of life.

In addition, even if the manufactured intraocular lens does not match the patient's vision, the degree of freedom of lens selection is high because it is possible to adjust the diopter at the surgical site or after surgery.

In addition, there are people who prefer to see close up well and people who prefer to see far away well depending on the person, and the vision can be adjusted even after the surgery according to the patient's preference, thereby improving the patient's satisfaction.

In addition, in the case of a patient with astigmatism, the patient's astigmatic axis and the astigmatic axis of the intraocular lens can be corrected to match after surgery, which improves the patient's satisfaction.

It should be understood that the effects of the present disclosure are not limited to the above-described effects, and include all effects inferable from a configuration of the invention described in detailed descriptions or claims of the present disclosure.

Although embodiments of the present disclosure have been described, the spirit of the present disclosure is not limited by the embodiments presented in the specification. Those skilled in the art who understand the spirit of the present disclosure will be able to easily suggest other embodiments by adding, changing, deleting, or adding components within the scope of the same spirit, but this will also be included within the scope of the spirit of the present disclosure.

What is claimed is:

1. An intraocular lens (IOL) with an adjustable diopter after surgery, comprising:
a haptic with a cylindrical receptacle, wherein one or more fixtures are configured to extend outwardly on an outer surface of the receptacle;
an optic mounted within the receptacle of the haptic, and configured to refract a light, wherein a thread is formed on an inner circumferential surface of the receptacle, and the optic is configured such that an outer circumferential surface thereof engages with the thread and rotates to be able to move forward or backward in a direction in which the light is incident in the receptacle; and
one or more magnetic members provided externally in at least one location at a periphery of the optic to rotate the optic by magnetic force.

2. The IOL of claim 1, wherein the magnetic member is a magnet or a magnet coated thereon.

3. The IOL of claim 1, wherein the magnetic member is a magnetic substance or a magnetic substance coated thereon.

4. The IOL of claim 1, wherein the magnetic member is configured to have a projected protrusion shape.

5. The IOL of claim 1, further comprising: a scale configured to indicate an angle by which the optic is rotated.

6. The IOL of claim 5, wherein the scale includes:
a reference scale formed on the optic, and rotated together according to rotation of the optic to become a reference point; and
rotation scales formed at a predetermined interval on a rim of the receptacle of the haptic to face the reference scale.

7. The IOL of claim 6, wherein the rotation scale is formed at intervals of 60 degrees.

8. The IOL of claim 1, wherein the thread is configured such that the optic is moved forward or backward by 1 mm when rotated 180 degrees in a normal or reverse direction.

9. The IOL of claim 1, wherein the receptacle includes a stopper at a front end or a rear end thereof, and
the stopper is configured to extend inwardly of the receptacle to prevent the optic from being removed while rotating forward and backward.
